(19) United States
(12) Patent Application Publication
(10) Pub. No.: US 2026/0092244 A1
(43) Pub. Date: Apr. 2, 2026

(54) MICRO-GROOVE STRUCTURE FOR CELL CULTURE AND CELL CULTURE DEVICE

(71) Applicant: ADVANCED BIOMED INC., Carson City, NV (US)

(72) Inventors: I-Fang CHENG, Tainan City (TW); Chen-Yi LEE, Tainan City (TW); Jyun-Hong WANG, Tainan City (TW); Pei-Mei LU, Tainan City (TW); Yi-Ching CHIU, Tainan City (TW); Chao-Hong CHEN, Tainan City (TW); Meng-Yen TSAI, Tainan City (TW)

(21) Appl. No.: 19/337,567

(22) Filed: Sep. 23, 2025

Related U.S. Application Data
(60) Provisional application No. 63/700,311, filed on Sep. 27, 2024.

(30) Foreign Application Priority Data
Mar. 13, 2025 (TW) 114109307

(51) Int. Cl. C12M 1/32; C12M 1/00

(52) U.S. Cl. CPC C12M 23/12; C12M 23/34

(57) ABSTRACT

A micro-groove structure for cell culture includes a micro-groove body and a a microstructure unit. The micro-groove body is formed with a microculture space which has an opening and which is defined by a space-defining wall. The microstructure unit is formed on the micro-groove body and is located in the microculture space. The microstructure unit has microstructures which protrude from the space-defining wall into the microculture space. The microstructures cooperatively define a concave surface which tapers in a direction away from the opening of the microculture space and which is used to support cells. A cell culture device includes a substrate and cell culture well units. The cell culture well units are disposed in the substrate. Each of the cell culture well units has a main well body and at least one the micro-groove structure.

### CROSS-REFERENCE TO RELATED APPLICATION

This application claims priority to Taiwanese Invention Patent Application No. 114109307, filed on Mar. 13, 2025, and claims the benefit of U.S. Provisional Ser. No. 63/700,311 , filed on Sep. 27, 2024, the entire disclosures of which are incorporated by reference herein.

### FIELD

The disclosure relates to a micro-structure and a culture device, and more particularly to a micro-groove structure for cell culture and a cell culture device.

### BACKGROUND

A conventional cell culture method includes culturing cells in a culture flask or a Petri dish so that the cells adhere and grow on a flat surface of the culture flask or the Petri dish. The cultured cells are usually formed as a monolayer. Morphologies and functions of the cells cultured by the conventional method may be distinct from those of cells in living organisms.

In recent years, cell culture methods have gradually evolved from two-dimensional culture methods to three-dimensional culture methods so that cultured cells may aggregate and grow into a three-dimensional structure that simulate organ tissues, and may be used for performing various biomimetic tests, such as drug tests and cancer cell research, thereby promoting developments of regenerative medicine and medical research.

Conventional three-dimensional culture methods are generally categorized into scaffold-based methods and scaffold-free methods.

The scaffold-based method uses a biopolymer material with a three-dimensional mesh structure as a scaffold to simulate an extracellular matrix environment, allowing cells to attach and grow. However, the three-dimensional scaffold may limit cell imaging and hinder observation.

The scaffold-free method is performed in the absence of physical support during cell cultivation, and includes, e.g., magnetic levitation methods and surface-coated non-adherent culture methods. The magnetic levitation method involves treating the cells with magnetic nanoparticles and then applying a magnetic force to cause the cells to aggregate and grow in a culture medium. The magnetic levitation method requires firstly treating the cells with magnetic nanoparticles. The surface-coated non-adherent culture method involves forming a coating on a bottom surface (with a curved, conical or V-shaped design) of a culture well so as to reduce contact areas between cells and the bottom surface of the culture well, allowing the cells to be suspended and easily aggregated into a spherical shape. However, the surface-coated non-adherent culture method requires a special coating treatment on the bottom surface of the culture well, and coating materials thus used must be non-toxic and non-degradable.

From the above, the current three-dimensional culture methods still have certain inconveniences and room for improvement.

### SUMMARY

Therefore, an object of the disclosure is to provide a cell culture device that can alleviate at least one of the drawbacks of the prior art.

According to a first aspect of the disclosure, a micro-groove structure for cell culture includes a micro-groove body and a microstructure unit. The micro-groove body is formed with a microculture space which has an opening and which is defined by a space-defining wall. The microstructure unit is formed on the micro-groove body and is located in the microculture space. The microstructure unit has microstructures which protrude from the space-defining wall into the microculture space. The microstructures cooperatively define a concave surface which tapers in a direction away from the opening of the microculture space and which is used to support cells.

According to a second aspect of the disclosure, a cell culture device includes a substrate and cell culture well units. The cell culture well units are disposed separately in the substrate. Each of the cell culture well units has a main well body and at least one micro-groove structure. The main well body is recessed from and extends downwardly from a top surface of the substrate, and has an orifice at the top surface of the substrate. The at least one micro-groove structure is disposed in the main well body and is adapted to cell culture. Each of the at least one micro-groove structure has a micro-groove body and a microstructure unit. The micro-groove body is recessed from an inner recessed wall of the main well body and is formed with a microculture space which has an opening and which is defined by a space-defining wall. The microstructure unit is formed on the micro-groove body and is located in the microculture space. The microstructure unit includes microstructures which protrude from the space-defining wall into the microculture space. The microstructures cooperatively define a concave surface which tapers in a direction away from the opening of the microculture space and which is used to support cells.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a perspective view illustrating a first embodiment of a cell culture device according to the disclosure.

FIG. 2 is a fragmentary sectional view illustrating a cell culture well unit of the first embodiment.

FIG. 3 is a fragmentary perspective view illustrating a micro-groove structure of the first embodiment, which has microstructures of hexagonal columns.

FIG. 4 is a view similar to FIG. 3, illustrating a variant of the micro-groove structure of the first embodiment, which has microstructures of hexagonal tubes.

FIG. 5 is a view similar to FIG. 2, illustrating another configuration of the cell culture well unit of the first embodiment.

FIG. 6 is a fragmentary sectional view illustrating a configuration of the cell culture well unit of the first embodiment, in which the microstructures are hexagonal columns.

FIG. 7 is a perspective view illustrating an arrangement of the microstructures which are in the hexagonal columns.

FIG. 8 is a fragmentary perspective view illustrating that the microstructures of the micro-groove structure of the first embodiment are shaped as hexagonal tubes.

FIG. 9 is a plan view illustrating that the microstructures of the hexagonal tubes are connected to one another to form a honeycomb shape.

FIG. 10 is a view similar to FIG. 6, illustrating another configuration of the microstructures of the first embodiment.

FIG. 11 is a flow diagram illustrating that during cell cultivation using the micro-groove structure of the first embodiment, cells aggregate towards a bottom central region of a concave surface.

FIG. 12 is a fragmentary perspective view illustrating another configuration of the micro-groove structure of the first embodiment.

FIG. 13 is a fragmentary sectional view illustrating the configuration of FIG. 12.

FIG. 14 is a fragmentary sectional view illustrating another configuration of the cell culture well unit of the first embodiment.

FIG. 15 is a fragmentary perspective view illustrating the cell culture well unit of a second embodiment of the cell culture device according to the disclosure.

FIG. 16 is a fragmentary sectional view illustrating the cell culture well unit of FIG. 15.

FIG. 17 is a fragmentary sectional view illustrating the cell culture well unit of a third embodiment of the cell culture device according to the disclosure.

FIG. 18 is a fragmentary top view illustrating the cell culture well units of FIG. 17.

FIG. 19 is a fragmentary sectional view illustrating the cell culture well unit of a fourth embodiment of the cell culture device according to the disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

It should be noted herein that for clarity of description, spatially relative terms such as “top,” “bottom,” “upper,” “lower,” “on,” “above,” “over,” “downwardly,” “upwardly” and the like may be used throughout the disclosure while making reference to the features as illustrated in the drawings. The features may be oriented differently (e.g., rotated 90 degrees or at other orientations) and the spatially relative terms used herein may be interpreted accordingly.

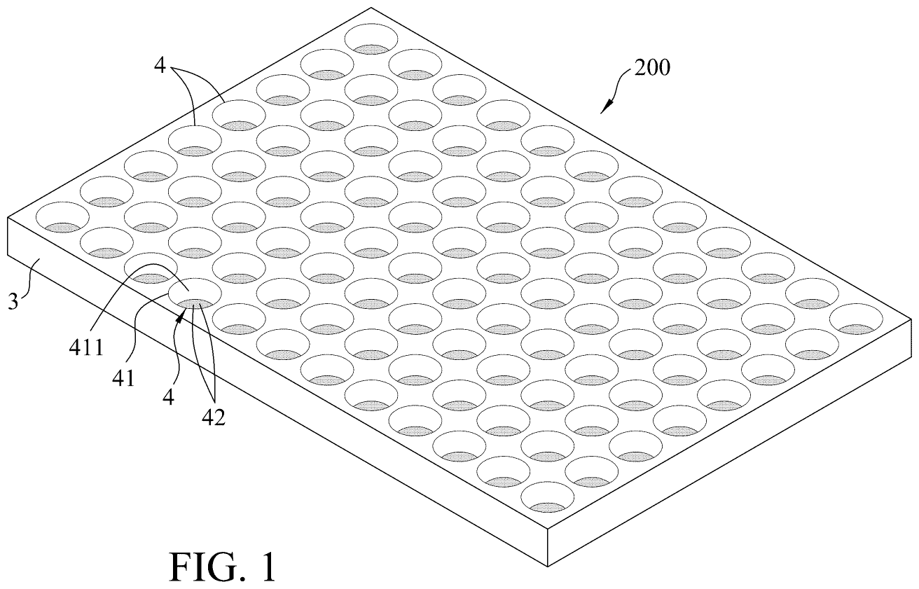

Referring to FIGS. 1 to 4, a first embodiment of a cell culture device 200 according to the disclosure is shown. The cell culture device 200 is adapted to accommodate a culture medium and is used for culturing cells 800. The cell culture device 200 includes a substrate 3 and cell culture well units 4 which are disposed separately in and recessed in the substrate 3.

Each of the cell culture well units 4 includes a main well body 41, micro-groove structures 42, and surface microstructures 45. The main well body 41 is recessed from and extends downwardly from a top surface of the substrate 3, has an orifice at the top surface of the substrate 3, and includes an inner recessed wall 410. The micro-groove structures 42 is disposed in the main well body 41. Specifically, the micro-groove structures 42 are disposed on and recessed separately from the inner recessed wall 410 of the main well body 41 and is adapted to cell culture. The surface microstructures 45 protrude from the inner recessed wall 410 toward the top surface of the substrate 3.

It should be noted that the micro-groove structures 42 and the surface microstructures 45 of the cell culture well units 4 all have micro-scale to nano-scale dimensions, and the configurations and dimensions shown in the figures of this disclosure are for illustrative purposes only and actual implementations are not limited thereto.

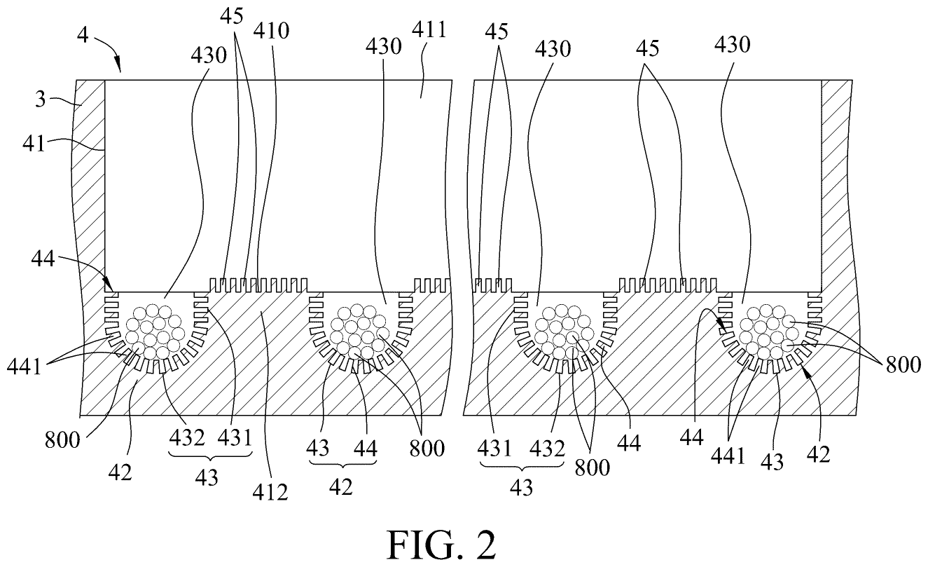

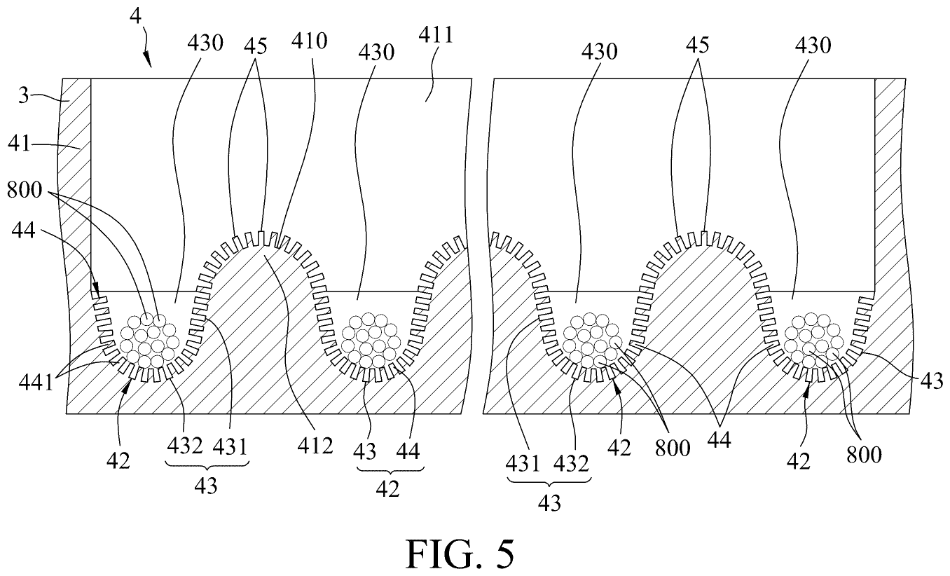

The main well body 41 defines a main culture space 411 which is in spatial communication with the micro-groove structures 42 and has the orifice. The inner recessed wall 410 defines a partition structure 412 which separates the micro-groove structures 42. A top surface of the partition structure 412 may be flat as shown in FIG. 2, or it may be arcuately convex as shown in FIG. 5. In the first embodiment, a diameter of the main culture space 411 of the main well body 41 of each of the cell culture well units 4 is 6 mm, but is not limited thereto during actual implementation.

The surface microstructures 45 protrude from the top surface of the partition structure 412, are perpendicular to the top surface of the partition structure 412, and extend uprightly as shown in FIG. 2 or extend obliquely as shown in FIG. 5. Moreover, in accordance with a shape of the partition structure 412, top ends of the surface microstructures 45 of each of the cell culture well units 4 are flush with one another as shown in FIG. 2, or cooperatively form a dome shape as shown in FIG. 5.

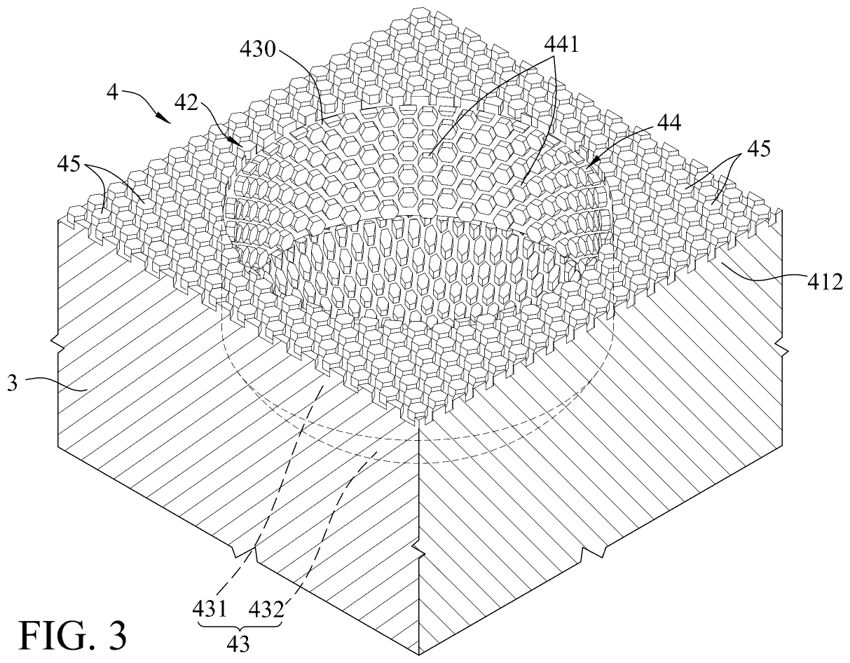

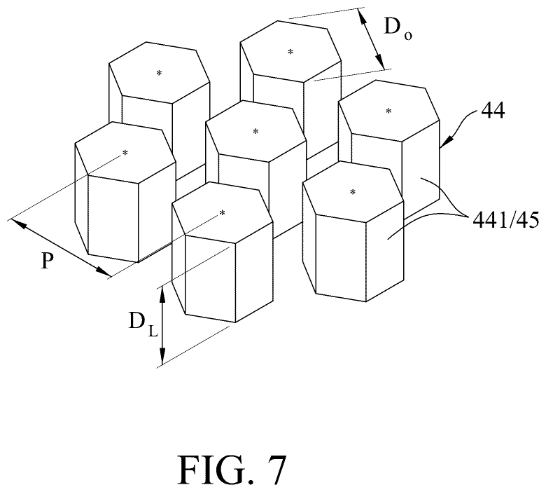

Referring to FIGS. 3 and 7, in the first embodiment, the surface microstructures 45 are polygonal columns which extend upwardly, are spaced apart from one another and are disposed uprightly on the partition structure 412. In the first embodiment, the surface microstructures 45 are designed to be hexagonal columns which are arranged in a honeycomb pattern. As shown in FIG. 7, a maximum width (diagonal diameter) of each of the surface microstructures 45 is D_{O}, and a height of each of the surface microstructures 45 is D_{L}. An aspect ratio (D_{L}/D_{O}) of the surface microstructures 45 is AR. A distance (P) between centers of two adjacent ones of the surface microstructures 45 ranges from 1.1D_{O} to 4D_{O}, and AR ranges from 0.1 to 15. In the first embodiment, D_{O} ranges from 200 nm to 3.5 μm, and D_{L} ranges from 20 nm to 10 μm. In other embodiments, each of the surface microstructures 45 may also be, for example, a triangular column, a quadrilateral column, a pentagonal column or a cylinder.

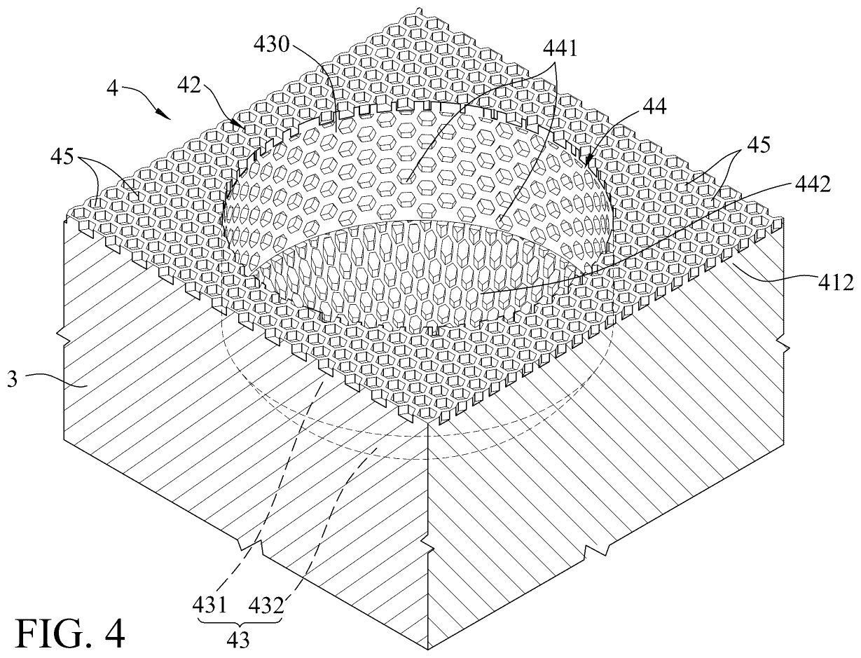

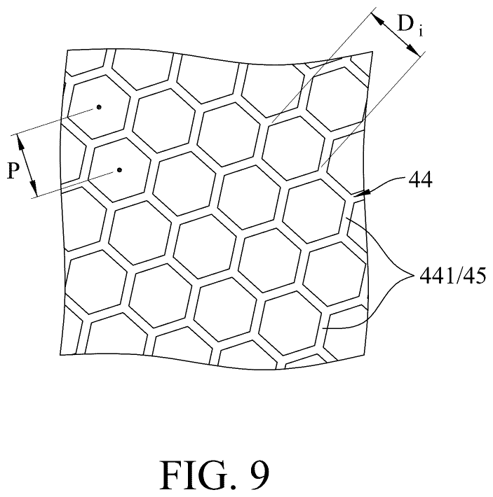

Referring to FIGS. 4 and 9, in certain embodiments of the disclosure, the surface microstructures 45 may also be hexagonal tubes which extend upwardly, are connected to one another and which are arranged in a honeycomb pattern. Each of the surface microstructures 45 of the hexagonal tubes has multiple walls which cooperatively define a hole. As shown in FIG. 9, a maximum width (diagonal diameter) of the hole of each of the surface microstructures 45 is Dᵢ, and a height of each of the surface microstructures 45 of the hexagonal tubes is D_{L}. An aspect ratio (D_{L}/Dᵢ) of the surface microstructures 45 is AR. A distance (P) between centers of two adjacent ones of the surface microstructures 45 of the hexagonal tubes ranges from 1.1Dᵢ to 4Dᵢ, and AR ranges from 0.1 to 15. In the first embodiment, Dᵢ ranges from 200 nm to 3.5 μm, and D_{L} ranges from 20 nm to 10 μm. In other embodiments, each of the surface microstructures 45 may also be, for example, a triangular tube, a quadrilateral tube, a pentagonal tube or a circular tube.

Since the micro-groove structures 42 may have the same structures, for the sake of convenience, only one of the micro-groove structures 42 will be described below for illustrative purposes.

Referring to FIGS. 2 and 3, the micro-groove structure 42 includes a micro-groove body 43 which is recessed from the inner recessed wall 410 of the main well body 41, and a microstructure unit 44 which is formed on the micro-groove body 43.

The micro-groove body 43 is formed with a microculture space 430 which has an opening and which is defined by a space-defining wall. The space-defining wall of the micro-groove body 43 has a loop-shaped peripheral wall 431 which defines the opening of the microculture space 430 and which extends vertically and downwardly from the inner recessed wall 410, and a recessed bottom wall 432 which extends downwardly from a bottom end of the loop-shaped peripheral wall 431 and which tapers in a direction away from the opening of the microculture space 430 to form a arc shape that opens upwardly. The loop-shaped peripheral wall 431 and the recessed bottom wall 432 of the space-defining wall cooperatively define the microculture space 430 which opens upwardly and which is in spatial communication with the main culture space 411.

In the first embodiment, the loop-shaped peripheral wall 431 of the micro-groove body 43 is in a circular shape which extends vertically as shown in FIG. 2. However, in other embodiments, the loop-shaped peripheral wall 431 may be in a cone shape which tapers in the direction away from the opening of the microculture space 430 as shown in FIG. 5. When the loop-shaped peripheral wall 431 is in a truncated cone shape, an angle of the loop-shaped peripheral wall 431 relative to a normal line of the top surface of the substrate 3 may range from 5 degrees to 89 degrees.

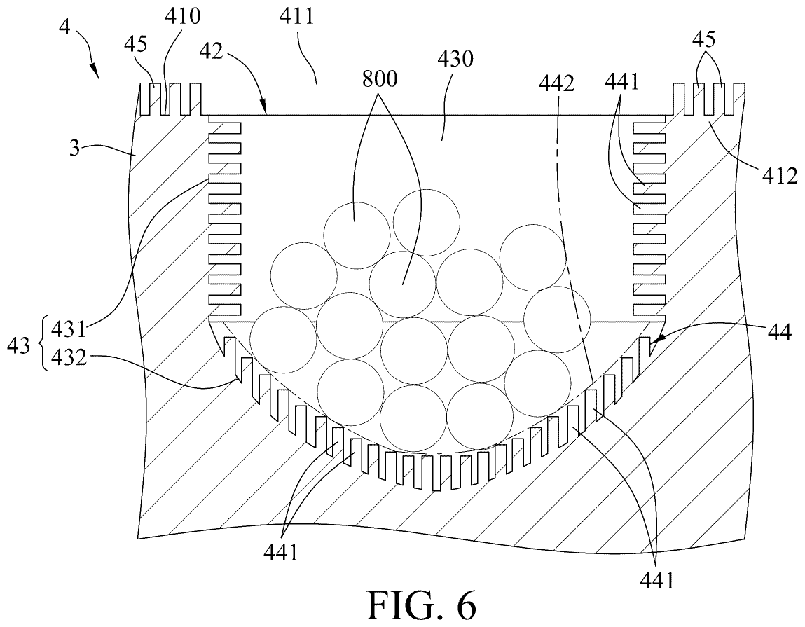

Referring to FIGS. 3 and 6, the microstructure unit 44 of the micro-groove structure 42 is located in the microculture space 430 and includes microstructures 441 which protrude from the loop-shaped peripheral wall 431 and the recessed bottom wall 432 into the microculture space 430 of the micro-groove body 43. The microstructures 441 which protrude from the loop-shaped peripheral wall 431 extend transversely as shown in FIG. 2 or 5. The microstructures 441 are disposed on, protrude from and extend upwardly from the recessed bottom wall 432.

The microstructures 441 which protrude from the recessed bottom wall 432 cooperatively define a concave surface 442 which tapers in the direction away from the opening of the microculture space 430 and which is used to support the cells 800. In the first embodiment, the concave surface 442 has an arc shape with a curvature ranging from 50 μm to 5 cm, and the concave surface 442 has an opening diameter or an opening width ranging from 10 μm to 1000 μm.

The microstructures 441 may have the two following configurations.

### Configuration 1: Polygonal Column

Referring to FIGS. 3, 6 and 7, each of the microstructures 441 is a polygonal column which has multiple side walls. The microstructures 441 are spaced apart from one another. Two adjacent ones of the microstructures 441 are arranged with one of the multiple side walls of one of the two adjacent ones of the microstructures 441 facing a respective one of the multiple side walls of another one of the two adjacent ones of the microstructures 441. The microstructures 441 which protrude from the recessed bottom wall 432 respectively have top surfaces that face the microculture space 430 and that cooperatively define the concave surface 442.

In this embodiment, each of the microstructures 441 is a hexagonal column. The microstructures 441 are arranged in a honeycomb pattern. A maximum width (diagonal diameter) of each of the microstructures 441 is D_{O}, and a height of each of the microstructures 441 is D_{L}. An aspect ratio (D_{L}/D_{O}) is AR. A distance (P) between centers of the two adjacent ones of the microstructures 441 ranges from 1.1D_{O} to 4D_{O}, and AR ranges from 0.1 to 15. In this embodiment, the maximum width of each of the microstructures 441 (D_{O}) ranges from 200 nm to 3.5 μm, and the height of each of the microstructures 441 (D_{L}) ranges from 20 nm to 10 μm. However, each of the microstructures 441 may be a triangular column, a quadrilateral column, an octagonal column or a cylinder.

### Configuration 2: Polygonal Tube

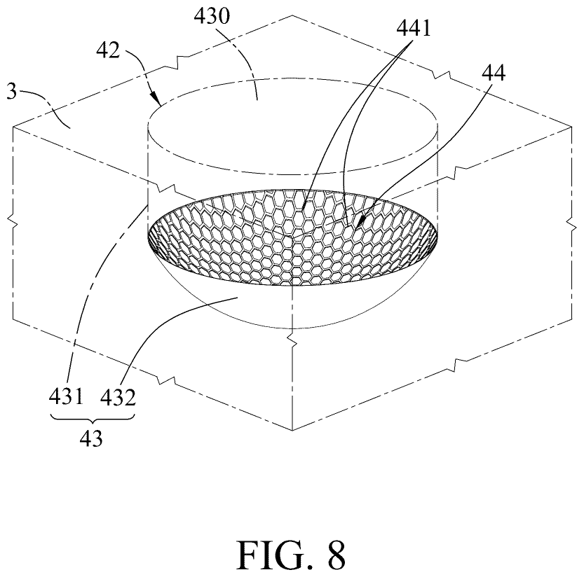

Referring to FIGS. 4, 8 and 9, each of the microstructures 441 is a polygonal tube. The microstructures 441 are connected to one another. The microstructures 441 which protrude from the recessed bottom wall 432 respectively have top surfaces that face the microculture space 430 and that cooperatively define the concave surface 442.

In this embodiment, each of the microstructures 441 is a hexagonal tube. The microstructures 441 are arranged in a honeycomb pattern. Each of the microstructures 441 has multiple side walls which cooperatively define a hole. A maximum width (diagonal diameter) of the hole of each of the microstructures 441 is Dᵢ, and a height of each of the microstructures 441 is D_{L}. An aspect ratio (D_{L}/Dᵢ) of the microstructures is AR. A distance (P) between centers of two adjacent ones of the microstructures 441 ranges from 1.1Dᵢ to 4Dᵢ, and AR ranges from 0.1 to 15. In this embodiment, the maximum width of the hole of each of the microstructures 441 (Dᵢ) ranges from 200 nm to 3.5 μm, and the height of each of the microstructures 441 (D_{L}) ranges from 20 nm to 10 μm. However, each of the microstructures 441 may be a triangular tube, a quadrilateral tube, a pentagonal tube or a circular tube.

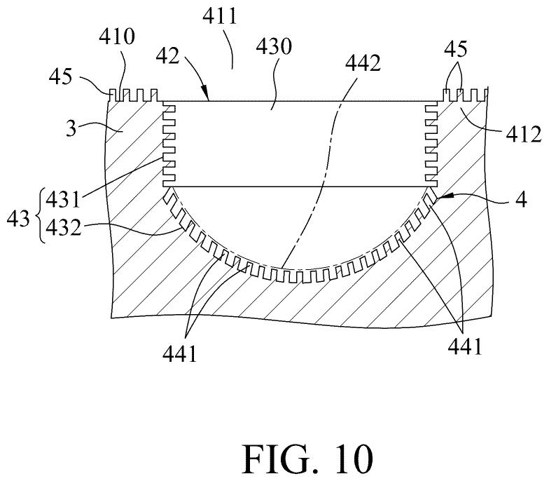

In the two abovementioned configurations, the microstructures 441 may protrude upwardly and vertically from the recessed bottom wall 432 as shown in FIG. 6. Alternatively, the microstructures 441 may protrude upwardly from the recessed bottom wall 432 and are perpendicular to the recessed bottom wall 432 as shown in FIG. 10; that is, some of the microstructures 441 may extend vertically and some of the microstructures 441 may extend obliquely.

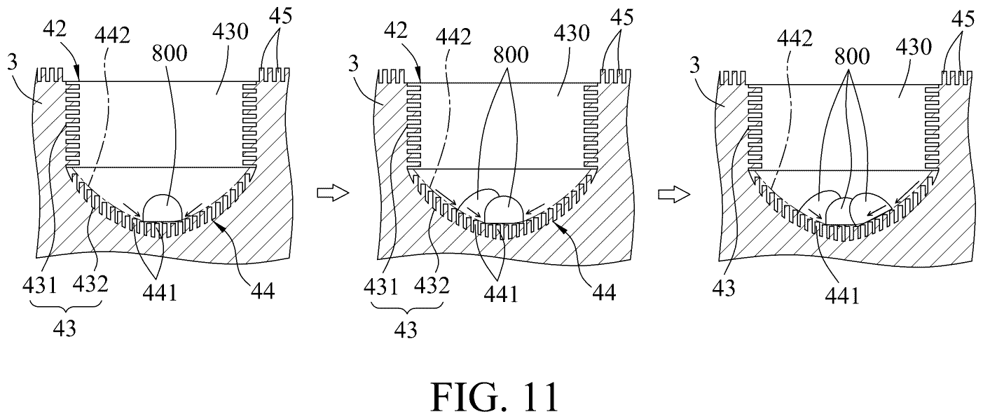

Referring to FIGS. 2, 6 and 11, through the two aforementioned structural designs of the micro-groove structure 42, when the culture medium containing the cells 800 is injected into the main well body 41, the cells 800 disperse and settle into the microculture space 430 defined by the micro-groove body 43. Since the concave surface 442 which is formed cooperatively by the micro-scaled or nano-scaled microstructures 441 is an arcuately concave surface with pores (constituted by the holes of the polygonal tubes or the spaces among the spaced-apart polygonal columns), the cells 800 are not easily attached to the concave surface 442. The cells 800 may slide and be concentrated towards a bottom central region of the concave surface 442 due to gravity. Through the structural designs, the cells 800 may proliferate at the bottom central region of concave surface 442 and aggregate into a spherical structure due to gravity.

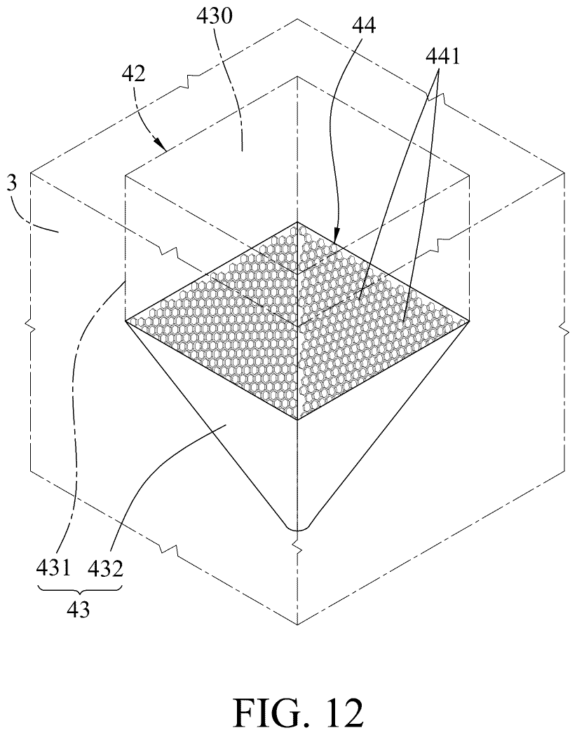

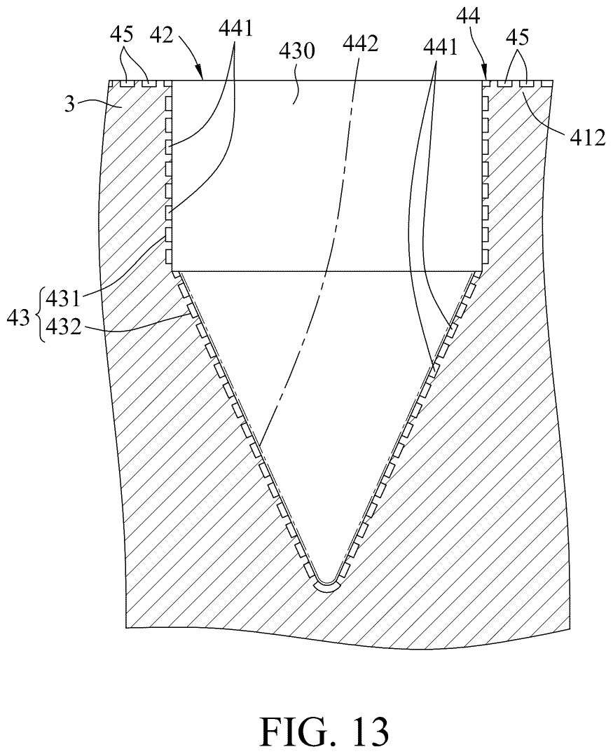

In the first embodiment, the loop-shaped peripheral wall 431 of the micro-groove body 43 is in a circular shape, and the recessed bottom wall 432 is in an arc shape as shown in FIG. 3. However, in other embodiments, the loop-shaped peripheral wall 431 of the micro-groove body 43 may be in an elliptical shape or a polygonal ring shape as shown in FIGS. 12 and 13, for example, but not limited to a triangular ring shape, a quadrilateral ring shape or a hexagonal ring shape. The shape of the recessed bottom wall 432 of the micro-groove body 43 corresponds to the shape of the loop-shaped peripheral wall 431, and may be a concave shape. The recessed bottom wall 432 may be shaped with a triangular pyramid, a quadrilateral pyramid or a hexagonal pyramid. The concave surface 442 which is formed by the microstructures 441 that are disposed on the recessed bottom wall 432 is also in a shape of tapered polygon. An angle of the concave surface 442 relative to the normal line of the top surface of the substrate 3 may range from 10 degrees to 75 degrees.

It should be noted that, each of the cell culture well unit 4 may have a plurality of the micro-groove structures 42 (e.g., fifty-five micro-groove structures 42) disposed in the main well body 41. However, in some embodiments, each of the cell culture well unit 4 may have only one micro-groove structure 42 in the main well body 41. In some embodiments, some of the cell culture well unit 4 are each provided with one micro-groove structure 42, and some of the cell culture well unit 4 are each provided with a plurality of the micro-groove structure 42.

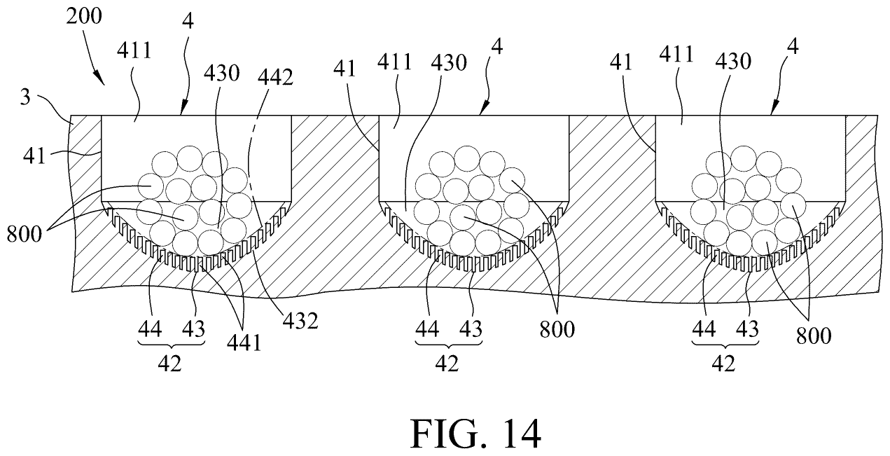

Referring to FIG. 14, in the embodiment where the cell culture well unit 4 has only one micro-groove structure 42 in the main well body 41, the micro-groove body 43 of the micro-groove structure 42 may not have the loop-shaped peripheral wall 431, and the recessed bottom wall 432 is directly connected to a bottom end of the main well body 41. The microculture space 430 is directly defined by the recessed bottom wall 432 such that an opening diameter or an opening width of the microculture space 430 is equal to the diameter of the main culture space 411.

Through this design, the cells 800 which are placed in the main well body 41 will directly settle into the microculture space 430 of the micro-groove body 43, and are guided by the microstructures 441 to aggregate and grow into a spherical structure at the bottom central region of the concave surface 442.

Moreover, with the surface microstructures 45 formed on the partition structure 412, when the top ends of the surface microstructures 45 are flush with one another as shown in FIG. 2, the cells 800 which settle on the surface microstructures 45 may not become easily attached to the surface microstructures 45, and may be encouraged to fall into the micro-groove structure(s) 42 by vibrating the cell culture device 200. When the top ends of the surface microstructures 45 cooperatively form a dome shape as shown in FIG. 5, the cells 800 which settle on the surface microstructures 45 are to be guided to fall into the micro-groove structure(s) 42. However, in certain embodiments, the surface microstructures 45 may be dispensed with.

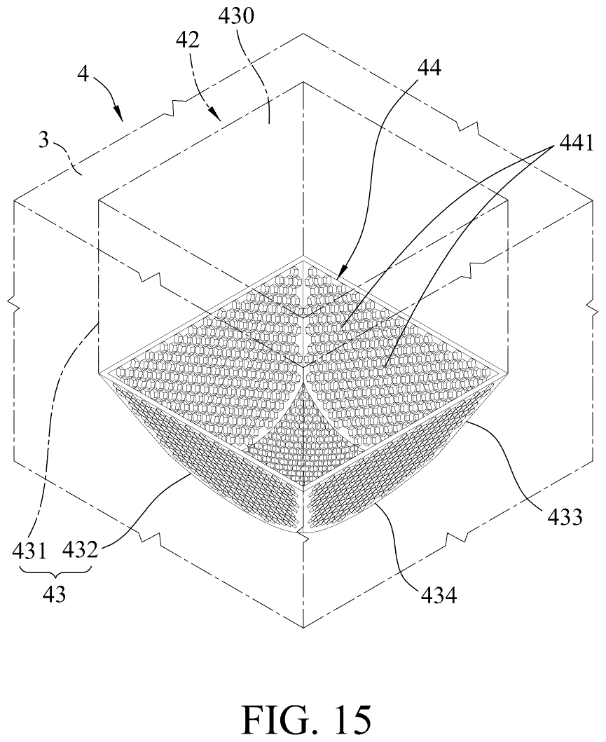

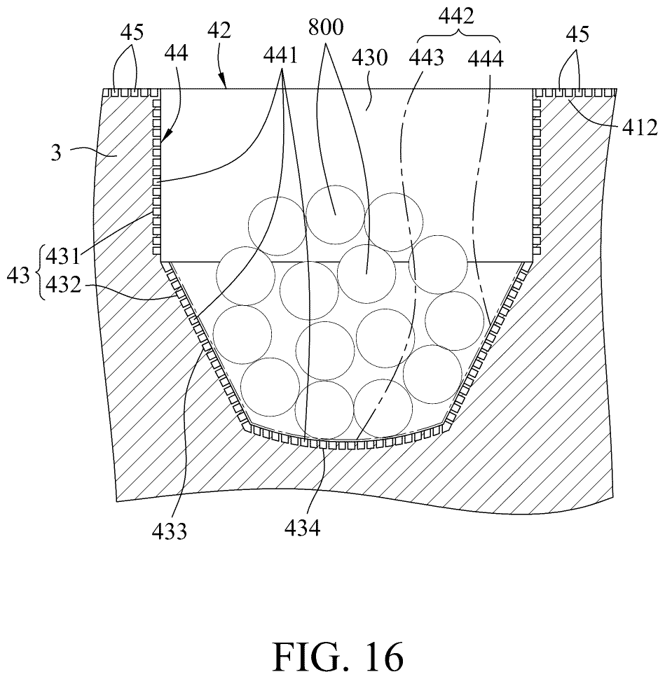

Referring to FIGS. 15 and 16, a second embodiment of the cell culture device 200 according to the disclosure is shown and is different from the first embodiment in terms of the structural design of the micro-groove body 43. For the sake of brevity, the following description only addresses the differences between the first embodiment and the second embodiment, and explanation is exemplified by only one micro-groove body 43.

In the second embodiment, the recessed bottom wall 432 of the micro-groove body 43 includes a tapered loop-shaped portion 433 which is connected to the bottom end of the loop-shaped peripheral wall 431 and which tapers in the direction away from the opening of the microculture space 430, and a recessed bottom portion 434 which is connected to a bottom end of the tapered loop-shaped portion 433 and which has an arc shape that opens upwardly. In some embodiments, the tapered loop-shaped portion 433 may be in a circular shape, an elliptical shape or a polygonal ring shape. The shape of the loop-shaped peripheral wall 431 corresponds to the shape of the tapered loop-shaped portion 433 of the recessed bottom wall 432, and is in a circular shape, an elliptical shape or a polygonal ring shape. An angle of the tapered loop-shaped portion 433 relative to a normal line of the top surface of the substrate 3 ranges from 10 degrees to 75 degrees.

The microstructures 441 which protrude from the tapered loop-shaped portion 433 and the recessed bottom portion 434 respectively have top surfaces that face the microculture space 430 and that cooperatively define the concave surface 442. Therefore, the concave surface 442 includes a collecting region 443 which is located at the recessed bottom portion 434 (i.e., a center region of the concave surface 442) and which has an arc shape that opens upwardly, and a guiding region 444 which is located at the tapered loop-shaped portion 433 and which extends outwardly and obliquely upwardly from a periphery of the collecting region 443 to form a loop shape (e.g., a polygonal ring shape). In certain embodiments, the microstructures 441 may be the same as the two configurations described in the first embodiment, i.e., the columns which are spaced apart from one another or the tubes which are connected to one another.

In certain embodiments, an angle of the guiding region 444 relative to the normal line of the top surface of the substrate 3 may range from 5 degrees to 135 degrees, and a length of the guiding region in a direction from the loop-shaped peripheral wall 431 to the recessed bottom portion 434 may range from 0.05 mm to 10 mm. The collecting region 443 (which corresponds to the recessed bottom portion 434) is in an arc shape which has a diameter that may range from 0.02 mm to 2.5 mm. That is, a diameter of the recessed bottom portion 434 ranges from 0.02 mm to 2.5 mm. In each of the micro-groove structures 42, a depth from the opening of the microculture space 430 of the micro-groove body 43 to a lowest point of the concave surface 442 (i.e., a lowest point of the collecting region 443) may range from 0.1 mm to 1.5 mm.

By virtue of the structural design of the recessed bottom wall 432 of the micro-groove body 43 and the structural design of the microstructure 441 which are disposed at the tapered loop-shaped portion 433 and the recessed bottom portion 434 of the recessed bottom wall 432, the cells 800 which fall into the microculture space 430 may be guided to the collecting region 443 of the concave surface 442, and aggregate at the collecting region 443 to grow into the spherical structure due to gravity.

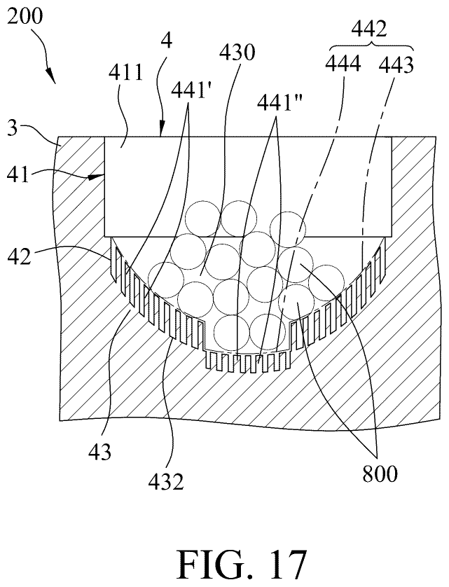

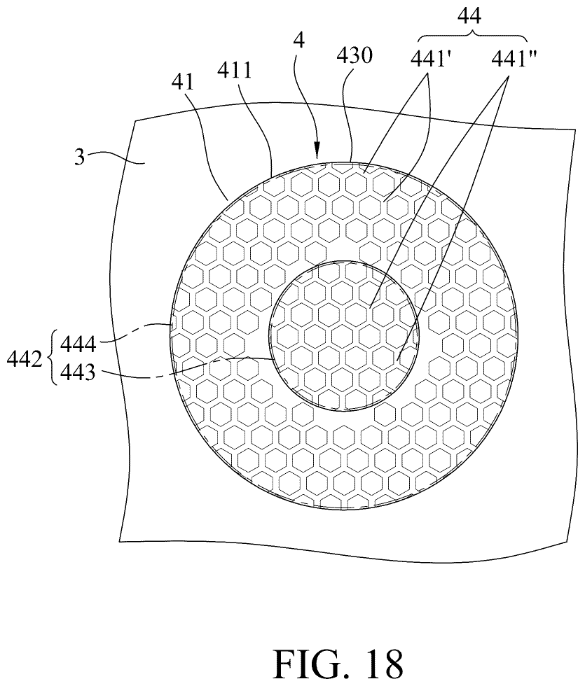

Referring to FIGS. 17 and 18, a third embodiment of the cell culture 200 according to the disclosure is different from the first embodiment in terms of a structural design of the cell culture well units 4. The following descriptions are provided using a single cell culture well unit 4 as an example.

In the third embodiment, only one micro-groove structure 42 is disposed in the main well body 41 of the cell culture well unit 4. The micro-groove body 43 of the micro-groove structure 42 only includes the recessed bottom wall 432. The recessed bottom wall 432 is connected to a bottom end of the main well body 41. An opening diameter or an opening width of the microculture space 430 which is defined by the recessed bottom wall 432 is equal to a diameter of the bottom end of the main culture space 411.

The concave surface 442 of the microstructures 441 of the microstructure unit 44 includes a collecting region 443 which is located at a center region of the concave surface 442, and a guiding region 444 which extends outwardly, curvedly and obliquely upwardly from a periphery of the collecting region 443 to form a loop shape. The collecting region 443 is farther away from the opening of the microculture space 430 than the guiding region 444. The microstructures 441 of the microstructure unit 44 are divided into first microstructures 441′ and second microstructures 441″. The first microstructures 441′ cooperatively define the collecting region 443. The second microstructures 441″ cooperatively define the guiding region 444. Top ends of the first microstructures 441′ are farther away from the opening of the microculture space 430 than top ends of the second microstructures 441″.

In the third embodiment, a diameter of the main culture space 411 is 6 mm, and a depth from an opening of the main culture space 411 (i.e., an orifice of the main well body 41) to a lowest point of the collecting region 443 is 10.5 mm. Each of the first microstructures 441′ is a hexagonal tube, and the first microstructures 441′ are connected to one another, are arranged in a honeycomb pattern and have top surfaces which cooperatively define the collecting region 443. Each of the second microstructures 441″ is a hexagonal column, and the second microstructures 441″ are spaced apart from one another, are arranged in a honeycomb pattern and have top surfaces which cooperatively define the guiding region 444. An area of the collecting region 443 may range from 0.01 mm² to 20 mm².

A maximum width of each of the second microstructure 441″ is D_{O}, and a distance (P) between centers of two adjacent ones of the second microstructures 441″ ranges from 1.1D_{O} to 4D_{O}. Each of the first microstructures 441′ has multiple side walls which cooperatively define a hole. A maximum width of the hole of each of the first microstructures 441′ is Dᵢ, and a distance (P) between centers of two adjacent ones of the first microstructures 441′ ranges from 1.1Dᵢ to 4Dᵢ.

As mentioned before, the D_{O} and Dᵢ in the aforesaid embodiments or configurations range from 200 nm to 3.5 μm. When the D_{O} and Dᵢ do not fall in this range (i.e., smaller than 200 nm or larger than 3.5 μm), the cells 800 will adhere tightly on the microstrucutres 441 (including the first microstructures 441′ and the second microstructures 441″) or the surface microstructures 45 rather than aggregate with one another to form the

### Spherical Structure.

In certain embodiments, the first microstructures 441′ and the second microstructures 441″ may be the same as the configurations described in the first embodiment. Moreover, the configuration of the first microstructures 441′ which defines the collecting region 443 may be interchangeable with the configuration of the second microstructures 441″ which defines the guiding region 444.

By virtue of the structural design of the micro-groove structure 42 of the cell culture well unit 4, when the culture medium containing the cells 800 is injected into the main well body 41, the cells 800 will settle directly into the micro-groove body 43 of the micro-groove structure 42. When the cells 800 fall into the microculture space 430 of the micro-groove body 43, since the guiding region 444 is a porous surface and is inclined inwardly and downwardly, the cells 800 will not be easily attached to the guiding region 444 and slide to the collecting region 443 due to gravity. Furthermore, since the collecting region 443 is also a porous surface which is inclined inwardly and downwardly, the cells 800 will also move towards and concentrate at a bottom central region of the collecting region 443. Through the structural design, the cells 800 may proliferate at the bottom central region of the collecting region 443 and aggregate into the spherical structure due to gravity.

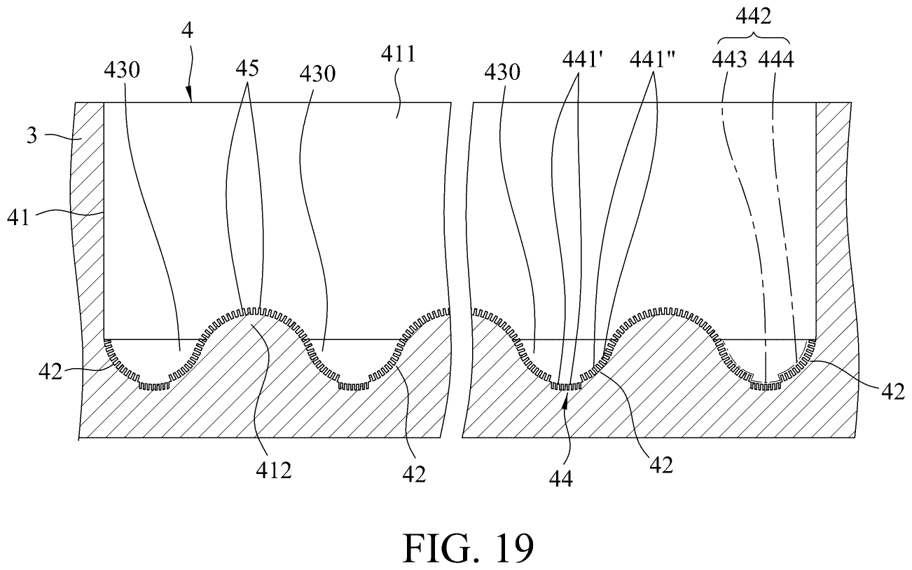

Referring to FIG. 19, a fourth embodiment of the cell culture device 200 according to the disclosure is different from the first embodiment in terms of a structural design of the cell culture well units 4. The following descriptions are provided using a single cell culture well unit 4 as an example.

In the fourth embodiment, the cell culture well unit 4 includes a plurality of the micro-groove structures 42 which have the structure shown in FIG. 17 of the third embodiment.

The top surface of the partition structure 412 is arcuately convex. The surface microstructures 45 are perpendicular to the top surface of the partition structure 412 and top ends of the surface microstructures 45 cooperatively form a dome shape.

The concave surface 442 of the microstructures 441 includes a collecting region 443 which is located at a center region of the concave surface 442, and a guiding region 444 which extends curvedly upwardly and outwardly from a periphery of the collecting region 443 to form a loop shape. The collecting region 443 is farther away from the opening of the microculture space 430 than the guiding region 444. The microstructures 441 of the microstructure unit 44 are divided into first microstructures 441′ and second microstructures 441″. The first microstructures 441′ cooperatively define the collecting region 443. The second microstructures 441″ cooperatively define the guiding region 444.

In summary, through the structural designs of the micro-groove body 43 and the microstructure unit 44 of the micro-groove structure 42, when the cells 800 fall into the micro-groove body 43, since the concave surface 442 which is formed cooperatively by the micro-scaled or nano-scaled columnar or tubular microstructures 441 is a porous surface, the cells 800 are less likely to be attached to the concave surface 442. That is, the concave surface 442 exhibits extremely low adhesion with the cells 800, so the cells 800 will slide downwardly to the bottom central region of the concave surface 442 (or the collecting region 443) along the concave surface 442 due to gravity. Thus, the cells 800 will proliferate at the bottom central region of the concave surface 442 (or the collecting region 443) and aggregate into the spherical structure.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A micro-groove structure for cell culture, comprising:
a micro-groove body which is formed with a microculture space that has an opening and that is defined by a space-defining wall; and
a microstructure unit which is formed on said micro-groove body and which is located in said microculture space, said microstructure unit including microstructures which protrude from said space-defining wall into said microculture space, said microstructures cooperatively defining a concave surface which tapers in a direction away from said opening of said microculture space and which is used to support cells.

2. The micro-groove structure for cell culture as claimed in claim 1, wherein each of said microstructures is a polygonal column which has multiple side walls, said microstructures being spaced apart from one another, two adjacent ones of said microstructures being arranged with one of said multiple side walls of one of said two adjacent ones of said microstructures facing a respective one of said multiple side walls of another one of said two adjacent ones of said microstructures, said microstructures respectively having top surfaces which face said microculture space and which cooperatively define said concave surface.

3. The micro-groove structure for cell culture as claimed in claim 2, wherein each of said microstructures is a hexagonal column, said microstructures being arranged in a honeycomb pattern, a maximum width of each of said microstructures being D_{O}, a distance between centers of said two adjacent ones of said microstructures ranging from 1.1D_{O} to 4D_{O}, said maximum width of each of said microstructures ranging from 200 nm to 3.5 μm.

4. The micro-groove structure for cell culture as claimed in claim 1, wherein each of said microstructures is a hexagonal tube, said microstructures being connected to one another and being arranged in a honeycomb pattern, said microstructures respectively having top surfaces which face said microculture space and which cooperatively define said concave surface, each of said microstructures having multiple side walls which cooperatively define a hole, a maximum width of said hole of each of said microstructures being Dᵢ, a distance between centers of two adjacent ones of said microstructures ranging from 1.1Dᵢ to 4Dᵢ, said maximum width of said hole of each of said microstructures ranging from 200 nm to 3.5 μm.

5. The micro-groove structure for cell culture as claimed in claim 1, wherein said concave surface includes a collecting region which is located at a center region of said concave surface, and a guiding region which extends outwardly and obliquely upwardly from a periphery of said collecting region to form a loop shape, said microstructures being divided into first microstructures and second microstructures, said first microstructures cooperatively defining said collecting region, said second microstructures cooperatively defining said guiding region, when said first microstructures are polygonal columns which are spaced apart from one another, said second microstructures are tubes which are connected to one another and which are arranged in a honeycomb pattern, and vice versa.

6. The micro-groove structure for cell culture as claimed in claim 5, wherein each of said first microstructures is a hexagonal tube, said first microstructures being connected to one another, being arranged in the honeycomb pattern and having top surfaces which cooperatively define said collecting region, each of said second microstructures being a hexagonal column, said second microstructures being spaced apart from one another, being arranged in the honeycomb pattern and having top surfaces which cooperatively define said guiding region, said collecting region being farther away from said opening of said microculture space than said guiding region.

7. The micro-groove structure for cell culture as claimed in claim 6, wherein a maximum width of each of said second microstructure is D_{O}, a distance between centers of two adjacent ones of said second microstructures ranging from 1.1D_{O} to 4D_{O}, each of said first microstructures having multiple side walls which cooperatively define a hole, a maximum width of said hole of each of said first microstructures being Dᵢ, a distance between centers of two adjacent ones of said first microstructures ranging from 1.1Dᵢ to 4Dᵢ.

8. The micro-groove structure for cell culture as claimed in claim 5, wherein an area of said collecting region ranges from 0.01 mm² to 20 mm².

9. The micro-groove structure for cell culture as claimed in claim 1, wherein said space-defining wall of said micro-groove body has a loop-shaped peripheral wall which defines said opening of said microculture space and which extends vertically, and a recessed bottom wall which extends downwardly from a bottom end of said loop-shaped peripheral wall and which opens upwardly, said microstructures of said micro-groove structure being disposed on and extending upwardly from said recessed bottom wall.

10. The micro-groove structure for cell culture as claimed in claim 2, wherein said space-defining wall of said micro-groove body has a loop-shaped peripheral wall which defines said opening of said microculture space and which extends vertically, and a recessed bottom wall which extends downwardly from a bottom end of said loop-shaped peripheral wall and which opens upwardly, said microstructures of said micro-groove structure being disposed on and extending upwardly from said recessed bottom wall.

11. The micro-groove structure for cell culture as claimed in claim 3, wherein said space-defining wall of said micro-groove body has a loop-shaped peripheral wall which defines said opening of said microculture space and which extends vertically, and a recessed bottom wall which extends downwardly from a bottom end of said loop-shaped peripheral wall and which opens upwardly, said microstructures of said micro-groove structure being disposed on and extending upwardly from said recessed bottom wall.

12. The micro-groove structure for cell culture as claimed in claim 4, wherein said space-defining wall of said micro-groove body has a loop-shaped peripheral wall which defines said opening of said microculture space and which extends vertically, and a recessed bottom wall which extends downwardly from a bottom end of said loop-shaped peripheral wall and which opens upwardly, said microstructures of said micro-groove structure being disposed on and extending upwardly from said recessed bottom wall.

13. The micro-groove structure for cell culture as claimed in claim 5, wherein said space-defining wall of said micro-groove body has a loop-shaped peripheral wall which defines said opening of said microculture space and which extends vertically, and a recessed bottom wall which extends downwardly from a bottom end of said loop-shaped peripheral wall and which opens upwardly, said microstructures of said micro-groove structure being disposed on and extending upwardly from said recessed bottom wall.

14. The micro-groove structure for cell culture as claimed in claim 6, wherein said space-defining wall of said micro-groove body has a loop-shaped peripheral wall which defines said opening of said microculture space and which extends vertically, and a recessed bottom wall which extends downwardly from a bottom end of said loop-shaped peripheral wall and which opens upwardly, said microstructures of said micro-groove structure being disposed on and extending upwardly from said recessed bottom wall.

15. The micro-groove structure for cell culture as claimed in claim 7, wherein said space-defining wall of said micro-groove body has a loop-shaped peripheral wall which defines said opening of said microculture space and which extends vertically, and a recessed bottom wall which extends downwardly from a bottom end of said loop-shaped peripheral wall and which opens upwardly, said microstructures of said micro-groove structure being disposed on and extending upwardly from said recessed bottom wall.

16. The micro-groove structure for cell culture as claimed in claim 8, wherein said space-defining wall of said micro-groove body has a loop-shaped peripheral wall which defines said opening of said microculture space and which extends vertically, and a recessed bottom wall which extends downwardly from a bottom end of said loop-shaped peripheral wall and which opens upwardly, said microstructures of said micro-groove structure being disposed on and extending upwardly from said recessed bottom wall.

17. The micro-groove structure for cell culture as claimed in claim 9, wherein said microstructures protrude upwardly and vertically from said recessed bottom wall.

18. The micro-groove structure for cell culture as claimed in claim 9, wherein said microstructures protrude upwardly from said recessed bottom wall and are perpendicular to said recessed bottom wall.

19. A cell culture device, comprising:
a substrate; and
cell culture well units which are disposed separately in said substrate, each of said cell culture well units including a main well body which is recessed from and extends downwardly from a top surface of said substrate and which has an orifice at said top surface of said substrate, and at least one micro-groove structure which is disposed in said main well body and which is adapted to cell culture, each of said at least one micro-groove structure including
a micro-groove body which is recessed from an inner recessed wall of said main well body and which is formed with a microculture space that has an opening and that is defined by a space-defining wall, and
a microstructure unit which is formed on said micro-groove body and is located in said microculture space, said microstructure unit including microstructures which protrude from said space-defining wall into said microculture space, said microstructures cooperatively defining a concave surface which tapers in a direction away from said opening of said microculture space and which is used to support cells.

20. The cell culture device as claimed in claim 19, wherein each of said microstructures is a polygonal column which has multiple side walls, said microstructures being spaced apart from one another, two adjacent ones of said microstructures being arranged with one of said multiple side walls of one of said two adjacent ones of said microstructures facing a respective one of said multiple side walls of another one of said two adjacent ones of said microstructures, said microstructures respectively having top surfaces which face said microculture space and which cooperatively define said concave surface.

21. The cell culture device as claimed in claim 20, wherein each of said microstructures is a hexagonal column, said microstructures being arranged in a honeycomb pattern, a maximum width of each of said microstructures being D_{O}, a distance between centers of said two adjacent ones of said microstructures ranging from 1.1D_{O} to 4_{O}, said maximum width of each of said microstructures ranging from 200 nm to 3.5 μm.

22. The cell culture device as claimed in claim 19, wherein each of said microstructures is a hexagonal tube, said microstructures being connected to one another and being arranged in a honeycomb pattern, said microstructures respectively having top surfaces which face said microculture space and which cooperatively define said concave surface, each of said microstructures having multiple side walls which cooperatively define a hole, a maximum width of said hole of each of said microstructures being Dᵢ, said distance between centers of two adjacent ones of said microstructures ranging from 1.1Dᵢ to 4Dᵢ, said maximum width of said hole of each of said microstructures ranges from 200 nm to 3.5 μm.

23. The cell culture device as claimed in claim 19, wherein said concave surface includes a collecting region which is located at a center region of said concave surface, and a guiding region which extends outwardly and obliquely upwardly from a periphery of said collecting region to form a loop shape, said microstructures being divided into first microstructures and second microstructures, said first microstructures cooperatively defining said collecting region, said second microstructures cooperatively defining said guiding region, when said first microstructures are polygonal columns which are spaced apart from one another, said second microstructures are tubes which are connected to one another and which are arranged in a honeycomb pattern, and vice versa.

24. The cell culture device as claimed in claim 23, wherein each of said first microstructures is a hexagonal tube, said first microstructures being connected to one another, being arranged in the honeycomb pattern and having top surfaces which cooperatively define said collecting region, each of said second microstructures being a hexagonal column, said second microstructures being spaced apart from one another, being arranged in the honeycomb pattern and having top surfaces which cooperatively define said guiding region, a maximum width of each of said second microstructure being D_{O}, a distance between centers of two adjacent ones of said second microstructures ranging from 1.1D_{O} to 4D_{O}, each of said first microstructures having multiple side walls which cooperatively define a hole, a maximum width of said hole of each of said first microstructures being Dᵢ, a distance between centers of two adjacent ones of said first microstructures ranging from 1.1Dᵢ to 4Dᵢ.

25. The cell culture device as claimed in claim 23, wherein an area of said collecting region ranges from 0.01 mm² to 20 mm².

26. The cell culture device as claimed in claim 19, wherein in each of said cell culture well units, said at least one micro-groove structure includes a plurality of micro-groove structures which are disposed on said inner recessed wall of said main well body.

27. The cell culture device as claimed in claim 20, wherein in each of said cell culture well units, said at least one micro-groove structure includes a plurality of micro-groove structures which are disposed on said inner recessed wall of said main well body.

28. The cell culture device as claimed in claim 21, wherein in each of said cell culture well units, said at least one micro-groove structure includes a plurality of micro-groove structures which are disposed on said inner recessed wall of said main well body.

29. The cell culture device as claimed in claim 22, wherein in each of said cell culture well units, said at least one micro-groove structure includes a plurality of micro-groove structures which are disposed on said inner recessed wall of said main well body.

30. The cell culture device as claimed in claim 23, wherein in each of said cell culture well units, said at least one micro-groove structure includes a plurality of micro-groove structures which are disposed on said inner recessed wall of said main well body.

31. The cell culture device as claimed in claim 24, wherein in each of said cell culture well units, said at least one micro-groove structure includes a plurality of micro-groove structures which are disposed on said inner recessed wall of said main well body.

32. The cell culture device as claimed in claim 25, wherein in each of said cell culture well units, said at least one micro-groove structure includes a plurality of micro-groove structures which are disposed on said inner recessed wall of said main well body.

33. The cell culture device as claimed in claim 26, wherein said space-defining wall of said micro-groove body has a loop-shaped peripheral wall which defines said opening of said microculture space and which extends vertically and downwardly from said inner recessed wall, and a recessed bottom wall which extends downwardly from a bottom end of said loop-shaped peripheral wall and which tapers in the direction away from said opening of said microculture space, said microstructures of each of said plurality of micro-groove structures being disposed on and extending upwardly from said recessed bottom wall.

34. The cell culture device as claimed in claim 33, wherein said inner recessed wall defines a partition structure which separates said plurality of micro-groove structures, a top surface of said partition structure being flat or arcuately convex.

35. The cell culture device as claimed in claim 34, wherein each of said cell culture well units further includes surface microstructures which protrude from said top surface of said partition structure.

36. The cell culture device as claimed in claim 35, wherein said surface microstructures are columns which extend upwardly, and are spaced apart from one another.

37. The cell culture device as claimed in claim 35, wherein said surface microstructures are tubes which extend upwardly, and are connected to one another.

38. The cell culture device as claimed in claim 35, wherein top ends of said surface microstructures of each of said cell culture well units are flush with one another or cooperatively form a dome shape.

39. The cell culture device as claimed in claim 33, wherein said microstructures protrude upwardly and vertically from said recessed bottom wall.

40. The cell culture device as claimed in claim 33, wherein said microstructures protrude upwardly from said recessed bottom wall and are perpendicular to said recessed bottom wall.

41. The cell culture device as claimed in claim 33, wherein said loop-shaped peripheral wall of said micro-groove body is in a circular shape, an elliptical shape or a polygonal ring shape, said recessed bottom wall of said micro-groove body extending downwardly from said bottom end of said loop-shaped peripheral wall to form a concave shape, said concave surface which is formed by said microstructures that are disposed on said recessed bottom wall being in a shape of a tapered polygon, an angle of said concave surface relative to a normal line of said top surface of said substrate ranging from 10 degrees to 75 degrees.

42. The cell culture device as claimed in claim 41, wherein in each of said plurality of micro-groove structures, a depth from said opening of said microculture space to a lowest point of said concave surface ranges from 0.1 mm to 1.5 mm.

43. The cell culture device as claimed in claim 33, wherein said loop-shaped peripheral wall of said micro-groove body is in a circular shape, an elliptical shape or a polygonal ring shape, said recessed bottom wall of said micro-groove body including a tapered loop-shaped portion which is connected to said bottom end of said loop-shaped peripheral wall and which tapers in the direction away from said opening of said microculture space, and a recessed bottom portion which is connected to a bottom end of said tapered loop-shaped portion and which is in an arc shape that opens upwardly, an angle of said tapered loop-shaped portion relative to a normal line of said top surface of said substrate ranging from 10 degrees to 75 degrees.

44. The cell culture device as claimed in claim 43, wherein a diameter of said recessed bottom portion ranges from 0.02 mm to 2.5 mm.
